(19) United States
(12) Patent Application Publication
(10) Pub. No.: US 2023/0293787 A1
(43) Pub. Date: Sep. 21, 2023

(54) HEART FAILURE RISK MONITORING AND HEMOGLOBIN LEVEL PREDICTION FOR HEMODIALYSIS SYSTEMS

(71) Applicant: Taipei Veterans General Hospital, Taipei City (TW)

(72) Inventors: Der-Cherng TARNG, Taipei City (TW); Shuo-Ming OU, Taipei City (TW); Yuan-Chia CHU, Taipei City (TW)

(73) Assignee: Taipei Veterans General Hospital, Taipei City (TW)

(21) Appl. No.: 18/124,164

(22) Filed: Mar. 21, 2023

Related U.S. Application Data
(60) Provisional application No. 63/321,863, filed on Mar. 21, 2022.

(51) Int. Cl. A61M 1/16; G16H 50/30; G16H 20/40; G16H 10/60

(52) U.S. Cl. CPC A61M 1/1601; G16H 10/60; G16H 20/40; G16H 50/30; A61M 2205/3334; A61M 2205/52

(57) ABSTRACT

Disclosed is a system comprising a dialysis machine for performing a dialysis treatment in a patient, and a computer system in communication with the dialysis machine. The computer system uses a trained ML model to generates a result that indicates the risk of the patient for developing heart failure during the dialysis treatment, and shows a warning sign on a display if the result indicates that the patient is at risk of heart failure.

### CROSS REFERENCE

This Non-provisional application claims the priority under 35 U.S.C. §119(e) on U.S. Pat. Provisional Application No. No. 63/321,863 filed on Mar. 21, 2022, the entire contents of which are hereby incorporated by reference.

### FIELD OF THE INVENTION

The present invention relates to the monitoring of risk of patients for developing heart failure in a dialysis treatment.

### BACKGROUND OF THE INVENTION

Hemodialysis (HD) patients are at high risk for developing dyslipidemia, left ventricular hypertrophy, and coronary artery disease, which may predispose them to heart failure (HF), which in turn leads to excess morbidity and mortality. End-stage renal disease (ESRD) per se represents an independent risk factor for HF, and both can coexist based on shared systemic diseases, such as diabetes mellitus or hypertension. Patients with ESRD at the time of initiation of dialysis therapy were often found to have prevalent HF. When compared with other hemodialysis patients, those with HF were independently associated with greater risks of adverse outcomes, including hospitalization or rehospitalization, morbidity and mortality, resulting in poor prognosis. According to the United States Renal Data System (USRDS) report, approximately 44% of hemodialysis patients have HF, and 13% of those patients have reduced ejection fraction (defined as a left ventricular ejection fraction (LVEF) < 40%). Although prompt diagnosis is mandatory before the initiation of medical and device-based therapies for HF, the diagnosis is often delayed because the signs and symptoms may be nonspecific in hemodialysis patients. Therefore, useful tools that can predict the risk of HF in hemodialysis patients are urgently needed.

HF is the most common cardiovascular complication of HD patients and is associated with adverse outcomes due to fluid overload or pulmonary congestion. Dry weight refers to the lowest post-dialysis weight tolerated with minimal signs or symptoms of hypovolemia or hypervolemia after a gradual change in weight following HD. Therefore, the accurate assessment of dry weight is important in HD patients for managing fluid status, reducing risks of hypertension and minimizing the burden on the cardiovascular system in HD patients.

In addition, among HD patients, erythropoiesis-stimulating agents (ESAs) are typically used to avoid severe anemia and reduce the need for blood transfusions, and therefore, treatment strategy to optimize hemoglobin target level may be needed and reduce the need for ESAs.

### SUMMARY OF THE INVENTION

In one aspect, the present invention provides a system comprising a dialysis machine for performing a dialysis treatment in a patient; and a computer system in communication with the dialysis machine, the computer system comprising: one or more processors; and a computer readable medium in communication with the one or more processors, the computer readable medium storing instructions that, when executed by the one or more processors, cause the computer system to perform: inputting a plurality of first features and a plurality of second features of the patient to a first trained machine learning (ML) model, wherein the plurality of first features is obtained during the dialysis treatment and the plurality of second features is obtained before the dialysis treatment, wherein the plurality of first features includes a total ultrafiltration volume value and a total ultrafiltration time value received from the dialysis machine, and wherein the plurality of second features includes a predictive value of pulmonary edema based on chest radiographic images, a Charlson comorbidity index value, a value of serum albumin level, a value of mean body surface area, a value of blood potassium level, and a value of predictive dry weight; generating, using the first trained ML model, a result that indicates the risk of the patient for developing heart failure during the dialysis treatment; and if the result indicates that the patient is at risk of heart failure, showing a warning sign on a display.

In some embodiments, the predictive value of pulmonary edema is generated using a second trained ML model with chest radiographic images of the patient as an input.

In some embodiments, the system of the present invention further comprises a time-series database which is connected to the computer system and stores real-time streaming intradialysis data received from the dialysis machine.

In some embodiments, at least part of the real-time streaming intradialysis data is shown on the display.

In some embodiments, the first trained ML model is trained using the following data: intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, arterial pressure, venous pressure, and transmembrane pressure, and predialysis data including demographic data, underlying comorbidities, containment medications and laboratory data.

In some embodiments, the value of predictive dry weight using a third trained ML model.

In some embodiments, the third trained ML model is trained using the following data: intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, arterial pressure, venous pressure, and transmembrane pressure, and predialysis data including demographic data, underlying comorbidities, containment medications and laboratory data.

In another aspect, the present invention provides one or more computer readable memories storing information to enable a computing device to perform a process comprising: inputting a plurality of first features and a plurality of second features of the patient to a first trained machine learning (ML) model, wherein the plurality of first features is obtained during the dialysis treatment and the plurality of second features is obtained before the dialysis treatment, wherein the plurality of first features includes a total ultrafiltration volume value and a total ultrafiltration time value received from the dialysis machine, and wherein the plurality of second features includes a predictive value of pulmonary edema based on chest radiographic images, a Charlson comorbidity index value, a value of serum albumin level, a value of mean body surface area, a value of blood potassium level, and a value of predictive dry weight; generating, using the first trained ML model, a result that indicates the risk of the patient for developing heart failure during the dialysis treatment; and if the result indicates that the patient is at risk of heart failure, showing a warning sign on a display.

In some embodiments, at least part of the real-time streaming intradialysis data is shown on the display.

In some embodiments, the first trained ML model is trained using the following data: intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and predialysis data including demographic data, underlying comorbidities, containment medications and laboratory data.

In some embodiments, the value of predictive dry weight using a third trained ML model.

In some embodiments, the third trained ML model is trained using the following data: intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and predialysis data including demographic data, underlying comorbidities, containment medications and laboratory data.

In a further aspect, the present invention provides a system comprising a dialysis machine for performing a dialysis treatment in a patient; and a computer system in communication with the dialysis machine, the computer system comprising: one or more processors; and a computer readable medium in communication with the one or more processors, the computer readable medium storing instructions that, when executed by the one or more processors, cause the computer system to perform: inputting a plurality of first features and a plurality of second features of the patient to a first trained machine learning (ML) model, wherein the plurality of first features is obtained during the dialysis treatment and the plurality of second features is obtained before the dialysis treatment, wherein the plurality of first features includes arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, transmembrane pressure, and wherein the plurality of second features includes serum iron level, total iron binding capacity (TIBC), and transferrin saturation percentage (TSAT); generating, using the first trained ML model, a predicted hemoglobin level of the patient during the dialysis treatment; and showing the predicted hemoglobin level on a display.

In some embodiments, the system further comprises a time-series database which is connected to the computer system and stores real-time streaming intradialysis data received from the dialysis machine. In some embodiments, at least part of the real-time streaming intradialysis data is shown on the display.

In some embodiments, the first trained ML model is trained using the following data: intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and predialysis data including demographic data, underlying comorbidities, containment medications and laboratory data.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows randomly selected examples of the prediction probabilities and heatmaps for the chest X-rays from a testing dataset;

FIG. 2 shows feature importance values of predictive models for heart failure risk;

FIG. 3A illustrates a dashboard shown on a display;

FIG. 3B illustrates another dashboard shown on the display; and

FIG. 4 is a bubble plot illustrating the correlation between left ventricular ejection fraction (LVEF) as measured by echocardiography and predictive ability for heart failure by machine learning models.

### DESCRIPTION OF THE INVENTION

The following embodiments when read with the accompanying drawings are made to clearly exhibit the above-mentioned and other technical contents, features and effects of the present disclosure. As the contents disclosed herein should be readily understood and can be implemented by a person skilled in the art, all equivalent changes or modifications which do not depart from the concept of the present disclosure should be encompassed by the appended claims.

Unless otherwise stated, the following terms used in this application, including the specification and claims, have the definitions given below.

As used in the specification and the appended claims, the singular forms “a,” “an” and “the” include plural referents unless the context clearly dictates otherwise. In this application, the use of “or” or “and” means “and/or” unless stated otherwise. Furthermore, use of the term “including” as well as other forms, such as “include”, “includes,” and “included,” is not limiting.

In one aspect, the present invention provides a system comprising a dialysis machine and a computer system. The dialysis machine is used for performing a dialysis treatment in a patient, and the computer system is in communication with the dialysis machine.

The system of the present invention may comprise multiple dialysis machines for performing dialysis treatments for multiple patients simultaneously.

In general, the computer system comprises one or more processors and a computer readable medium in communication with the one or more processors. The computer readable medium stores instructions that, when executed by the one or more processors, cause the computer system to perform steps described herein.

In another aspect, the present invention provides one or more computer readable memories storing information to enable a computing device to perform a process as described herein.

The computer system or computing device may first receive intradialysis data of the patient from the dialysis machine and predialysis data of the patient from a database, which may be used to train machine learning (ML) models described herein.

An ML model used in the present invention may be a deep learning network or other machine learning model.

The intradialysis data is collected during the dialysis treatment and may include arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, transmembrane pressure, or a combination thereof.

The intradialysis data may also include vital signs, such as systolic blood pressure, diastolic blood pressure, pulse rate, or body temperature.

On the other hand, the predialysis data is collected or determined before the dialysis treatment and may include the patient’s demographic data (e.g., age, gender, etc.), underlying comorbidities, containment medications and laboratory data.

The laboratory data may include blood test data. The blood test data may include blood levels of urea nitrogen, creatinine, white blood cells, hemoglobin, platelets, albumin, sodium, potassium, chloride, aspartate aminotransferase, alanine aminotransferase, urea nitrogen, total CO2, bilirubin, calcium, glucose, creatine kinase, lipase, troponin I, or a combination thereof. The laboratory data may also include serum iron level, total iron binding capacity (TIBC), and transferrin saturation percentage (TSAT).

The term “data” as used herein may refer to a statistical value of data. The statistical value includes but is not limited to a mean value, a maximum value, a minimum value, a median value, or a quartile value. For example, body temperature may refer to mean body temperature over a period of time, and blood pressure may refer to average real variability (ARV) of blood pressure.

According to the present invention, the predictive value of pulmonary edema may be generated using a trained ML model with chest radiographic images of the patient as an input. In one preferred embodiment, the ML model is a VGG16 network.

In some embodiments, the value of predictive dry weight is also generated or determined by a trained ML model.

The term “mean body surface area” as used herein refers to a value calculated by the Mosteller formula, sqrt{[height (cm) x weight (kg)]/3600}. On average, the mean body surface area is 1.9-2.1 m² for males and 1.6-1.8 m² for females.

The term “predictive dry weight” refers to a predicted optimal dry weight for a hemodialysis patient, usually estimated by comparing the previous and current measurements of dry weight (predialysis body weight). Predictive dry weight is a function of a patient’s body weight, blood pressure, biochemical indicators before and after dialysis, gender, age, etc. For example, a predictive dry weight may be estimated as (weight_1 + weight _2)/2, where weight_1 is the predialysis body weight of a patient measured in the last dialysis treatment, and weight_2 is the predialysis body weight of the patient measured in the present dialysis treatment.

Next, the computer system or computing device may extract a plurality of first features and a plurality of second features of the patient from the received intradialysis data and predialysis data, respectively, and input the plurality of first features and the plurality of second features to a trained ML model.

For heart failure risk prediction, the plurality of first features preferably includes a total ultrafiltration volume value and a total ultrafiltration time value, and the plurality of second features preferably includes a predictive value of pulmonary edema based on chest radiographic images, a CCI value, a value of serum albumin level, a value of mean body surface area, a value of blood potassium level, and a value of predictive dry weight.

For hemoglobin level prediction, the he plurality of first features includes arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, transmembrane pressure, and the plurality of second features includes serum iron level, total iron binding capacity (TIBC), and transferrin saturation percentage (TSAT).

In some embodiments, the hemoglobin level may be predicted as c*(predicted serum ferritin level)*(predicted TSAT), where the predicted serum ferritin level is generated using a trained ML model, the predicted TSAT is generated by a trained ML model, and c is a constant.

The computer system or computing device uses a trained ML model to generate a result that indicates the risk of the patient for developing heart failure during the dialysis treatment, or to generate a predicted hemoglobin level of the patient during the dialysis treatment.

In some embodiments, the trained ML model may first generate an output that characterizes the risk of the patient for developing heart failure during the dialysis treatment, the computer system or computing device generates, based on the output, a result comprising a numerical value representing a probability that the patient will develop heart failure, a risk level of the patient for developing heart failure, or a combination thereof.

Preferably, the computer system or computing device comprises or is connected to a display or display screen.

If the result indicates that the patient is at risk of heart failure, the computer system or computing device shows on the display or display screen a warning sign to inform, for example, medical personnel. For example, the warning sign may be a message “HF Danger” in red fonts shown in a column corresponding to a specific patient.

On the other hand, the predicted hemoglobin level may also be shown on the display or display screen.

According to certain embodiments of the present invention, the ML model used in generating said result is trained using data including intradialysis data and predialysis data as described above.

The ML model used in generating said result may be based on an algorithm selected from the group consisting of logistic regression, linear regression, generalized linear models, nonlinear regression, ordinary least squares regression, partial least squares regression, quartile regression, random forest, gradient boosting, support vector machines, and neural networks. Preferably, the ML model used in generating said result is based on a random forest algorithm.

According to certain embodiments of the present invention, the ML model used in generating or determining the predictive dry weight is trained using data including intradialysis data and predialysis data as described above.

In some embodiments, the computer system or computing device acquires the intradialysis data and predialysis data from the dialysis machine and a database, respectively, through performing an extract, transform, load (ETL) process.

Further, the display or display screen may be used to show real-time streaming intradialysis data of the patient.

In some embodiments, the system of the present invention further comprises a time-series database. The time-series database is in communication with the computer system or computing device and is used for storing real-time streaming intradialysis data received from the dialysis machine. The computer system or computing device may acquire some of the important intradialysis data from the time-series database and show them on the display.

### EXAMPLES

### 1. Methods

### 1.1 Data Acquisition

Data were acquired from patients with end-stage renal disease (ESRD) who underwent regular hemodialysis in the Division of Nephrology, Department of Internal Medicine at Taipei Veterans General Hospital from July 2017 to July 2019. The comprehensive data of the hemodialysis patients were extracted from the Big Data Center, which included all medical records, pharmacy orders, laboratory results, and chest radiogram images from all inpatient, outpatient, and emergency services.

The clinical features were derived from predialysis and intradialytic data. The medical devices were set up to gather continuous stream data based on time-series labels and connected to the analysis server. The analysis server includes the research database of the Big Data Center that stores time-serial continuous data, an artificial intelligence module that establishes machine learning models, and a medical decision support module that visualizes the results of the best-performing models and provides warnings on a visualized dashboard.

### 1.2 Input Features - Predialysis Data

Predialysis features included demographic data, underlying comorbidities, containment medications and laboratory data extracted from within the database. Category features such as demographic data, underlying comorbidities, and containment medications were encoded as binary variables using medical diagnostic coding ICD-10. Patient demographic data included age, sex, body weight, body height, body mass index, and body surface area. Underlying comorbidities were identified by using the International Classification of Diseases diagnostic codes, and the Charlson Comorbidity Index (CCI) score was used to determine overall systemic health. Containment medications with corresponding Anatomical Therapeutic Chemical (ATC) codes were extracted and classified based on the ATC classification system recommended by the World Health Organization. Laboratory values included white blood cell count, hemoglobin, platelet count, blood urea nitrogen, creatinine, sodium, potassium, chloride, calcium, phosphate, albumin, total protein, aspartate aminotransferase, alanine aminotransferase, alkaline phosphatase, gamma-glutamyl transferase, total bilirubin, glucose, glycated hemoglobin, cholesterol, low-density lipoprotein cholesterol, high-density lipoprotein cholesterol, triglyceride, uric acid, creatine kinase, lactate dehydrogenase, iron, total iron binding capacity, ferritin, intact parathyroid hormone, C-reactive protein and Kt/V. The mean, maximum and minimum values of each laboratory variable were calculated, and the features of the laboratory data were transformed to a combined boxplot of features after data processing for model prediction.

### 1.3 Input Features - Intradialysis Data

Intradialysis features were extracted from the massive data generated by the dialysis machines, and these included arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, arterial pressure, venous pressure, transmembrane pressure, dialysate pressure, dialysis membrane pressure, pigment clearance rate (Kt/V), dialysate flow amount, temperature, pH, ammonia, nitrogen, chloride ion concentration, etc. of dialysate, vital signs such as body temperature, heart rate, blood oxygen saturation, respiratory rate, etc., treatment time, and blood temperature. Within a 3-4-hour dialysis session, the intradialytic features of the stream data from the dialysis machines were collected every minute. To concatenate the stream data from dialysis machines with the predialytic data, we built a time series database to process these stream data.

### 1.4 Input Features-Chest Radiography

We used a deep convolutional neural network model, VGG-16, to determine whether patients had pulmonary edema from chest radiograph images obtained prior to hemodialysis sessions. The pipeline of the deep convolutional neural network of our study includes chest image preprocessing and then classifies images as “normal” or “pulmonary edema” through the VGG-16 method. The VGG-16 method consists of four main building blocks: convolutional layers, pooling layers, fully connected layers, and the softmax output layer. All the layers of the VGG-16 method were fine-tuned by performing training for 200 epochs using a stochastic gradient descent (SGD) optimizer (a batch size of 64 images, a learning rate of 0.0001, and a dropout of 0.5). The log loss for the training and validation datasets was evaluated. After we trained the model, we generated a heatmap that demonstrates the regions in the image that were important for classification. The heatmap results allow us to gain insight into the “black box” nature of the model as well as to point out the locations of pulmonary edema from chest radiography. As an additional predialytic feature, we incorporated pulmonary edema predictive values from chest radiography into our heart failure (HF) prediction models as continuous variables.

### 1.5 Class Labels

Hemodialysis patients routinely underwent annual echocardiography at our hospital. Based on the measurements of echocardiography, a left ventricular ejection fraction (LVEF) < 40% was considered a clinically relevant cutoff value to define abnormal LV function. LVEF was measured using apical two- and four-chamber views, and all echocardiograms were reviewed by expert cardiologists. If patients received multiple echocardiographs, we used the LVEF value closest to the date of hemodialysis in our analyses. HF was annotated as 1 if the LVEF was < 40% on the echocardiogram report and annotated as 0 if the LVEF was > 40%.

### 1.6 Data Collection and Processing

To execute and test the machine learning models, we used SAS and Python Visual Data Mining and Machine Learning to implement the machine learning models on SAS Viya for the comprehensive set of built-in environments and functions for computational, data mining, and machine learning. The machine learning algorithm involved the following steps: 1) data collection, processing, selection and imputing missingness; 2) featurization of the massive data stream from the dialysis machines; and 3) annotation of the events of outcomes in the training dataset. After data processing, the analytic dataset was randomly assigned into training (60%), validation (30%) and test (10%) and used the formed feature set in the machine learning model training framework.

### 1.7 Development of the Machine Learning Algorithms

From the model building and model assessment perspective, SAS Visual Data Mining and Machine Learning on SAS Viya enables the construction of automated machine learning models to select the best-performing model through the model comparison within a maximum of 60 minutes through the tree-based pipeline. Automated machine learning models of SAS Visual Data Mining and Machine Learning includes logistic regression, linear regression, generalized linear models, nonlinear regression, ordinary least squares regression, partial least squares regression, quartile regression, random forest, gradient boosting, support vector machines and neural networks. The accuracy of different machine learning models during the model training and validation stages was compared, and an independent test dataset was used to evaluate their performance. Based on the F1 score of the models, the best-performing model was selected as the champion model. We synchronously built a big data-based visualized champion model on the machine learning platform based on SAS Viya Artificial intelligence of things (AIoT) and a high-performance NVIDIA graphic processing unit (GPU) computing platform. The results of HF predictive values from the trained champion model are shown on the visualized Grafana dashboard for monitoring. Granana is an open-source multiplatform for analytics and interactive visualization that provides charts, graphs, and alerts connected to our time-serial streams of data and our machine learning results to generate visualized graphs. AI models were trained and validated using an NVIDIA DGX-1 server with a 20-core Intel CPU, 8X NVIDIA V100 SMX2 32-GB GPU card, and 512 GB of available RAM.

### 1.8 Model Assessment and Statistical Analyses

To assess model performance, we calculated the area under the receiver operating characteristic curve (AUROC), accuracy, F1 score, misclassification rate, false positive rate, and false discovery rate of the predictive ability for HF. Accuracy =(TP+TN)/(TP+TN+FP+FN); false-positive rate = FP/(FP+TN) (TP: true positive; TN: true negative; FP: false positive; FN: false negative). Misclassification rate: (FP+FN)/(TP+TN+FP+FN). False discovery rate= FP/(TP+FP). The F1 score is a formula combining the precision and recall of the model, defined as the harmonic mean of the model’s precision and recall. To inspect the relative influence of important features (e.g., predialysis features and intradialytic stream data from dialysis machines, etc.) on machine learning models, we explored feature importance within machine models.

### 2. Results

### 2.1 Study Population

A total of 448 hemodialysis patients aged 20 years and older were included from 31 Jul. 2017 to 31 Jul. 2019. We randomly divided 448 patients into three groups: 269 patients (60%) were assigned to the training set, 134 patients (30%) were assigned to the testing set, and 44 patients (10%) were assigned to the validation set. There were 579,052 data records in the training dataset, 63,670 in the validation dataset, and 246,734 in the testing dataset, with a total of 889,456 data records in our analyses.

### 2.2 Chest Radiogram Heatmaps

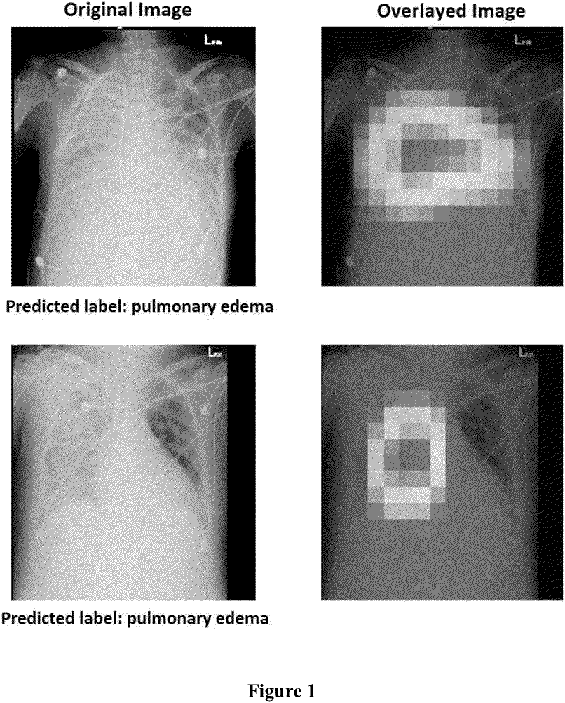

In our study, chest radiogram images extracted from the database were labeled and divided into “normal” and “pulmonary edema” images. FIG. 1 shows randomly selected examples of the prediction probabilities and heatmaps for the chest X-rays from the testing dataset. The original image is on the left, and its heatmap is on the right, with its prediction probability written below. Red areas on the heatmaps show important regions, according to the classification determined by the deep convolutional neural network model VGG-16. The learning rate is 0.001 and the performance of both the training and the validation improves significantly and is very stable.

### 2.3 Predictive Ability of the Machine Learning Models and Feature Importance Plots

Random forest (2) was selected as the champion model for HF prediction, with an accuracy and AUROC of 0.942 and 0.957, respectively (see Table 1 below). The F1 score, false positive rate, false discovery rate and misclassification rate of our predictive models were 0.842, 0.029, 0.132 and 0.058, respectively.

TABLE 1

Model performance in predicting risk for HF in hemodialysis patients

| | Accuracy | AUROC | F1 Score | False Positive Rate | False Discovery Rate | Misclassification Rate | Average Squared Error | Root Average Squared Error | Gini Coefficient | Log Loss |
|---|---|---|---|---|---|---|---|---|---|---|
| Random forest (1) | 0.894 | 0.972 | 0.775 | 0.124 | 0.354 | 0.106 | 0.080 | 0.283 | 0.943 | 0.244 |
| Supervised Learning (1) | 0.943 | 0.915 | 0.832 | 0.010 | 0.052 | 0.057 | 0.051 | 0.226 | 0.829 | 0.208 |
| Random forest (2)^{†} | 0.942 | 0.957 | 0.842 | 0.029 | 0.132 | 0.058 | 0.052 | 0.229 | 0.913 | 0.177 |
| Random forest (3) | 0.927 | 0.958 | 0.788 | 0.025 | 0.130 | 0.073 | 0.059 | 0.242 | 0.916 | 0.210 |
| Supervised Learning (2) | 0.884 | 0.914 | 0.750 | 0.124 | 0.366 | 0.116 | 0.085 | 0.291 | 0.829 | 0.276 |
| Gradient Boosting | 0.943 | 0.915 | 0.832 | 0.010 | 0.052 | 0.057 | 0.051 | 0.226 | 0.829 | 0.208 |

†Random forest (2) was selected as best-performing model based on the highest F1 score. Abbreviations: HF, heart failure; AUROC, area under the curve of receiver operating characteristic curve.

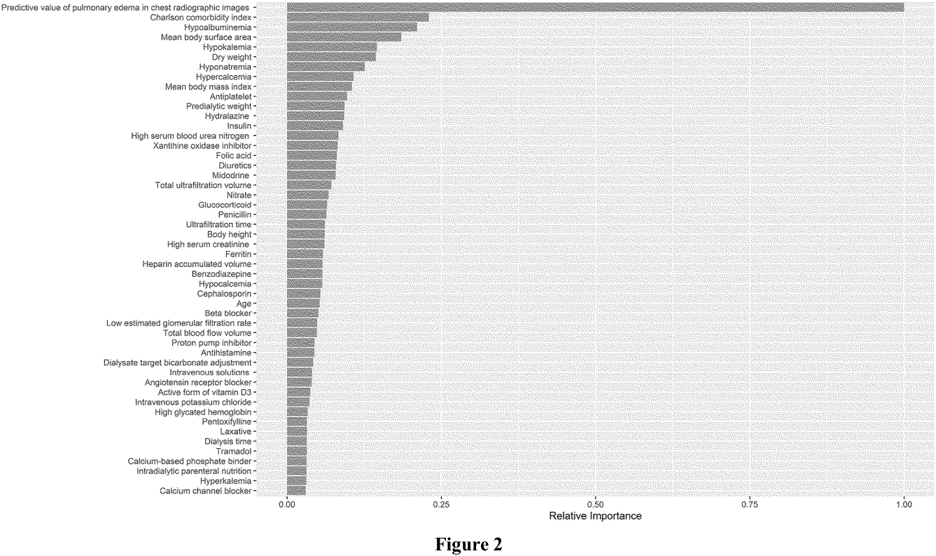

Feature importance values from our predictive models are shown in FIG. 2. Predialytic features, including the predictive value of pulmonary edema in chest radiographic images, CCI, hypoalbuminemia, mean body surface area, hypokalemia, and dry weight, were the most important features for the detection of HF. The most important intradialytic features included total ultrafiltration volume and ultrafiltration time.

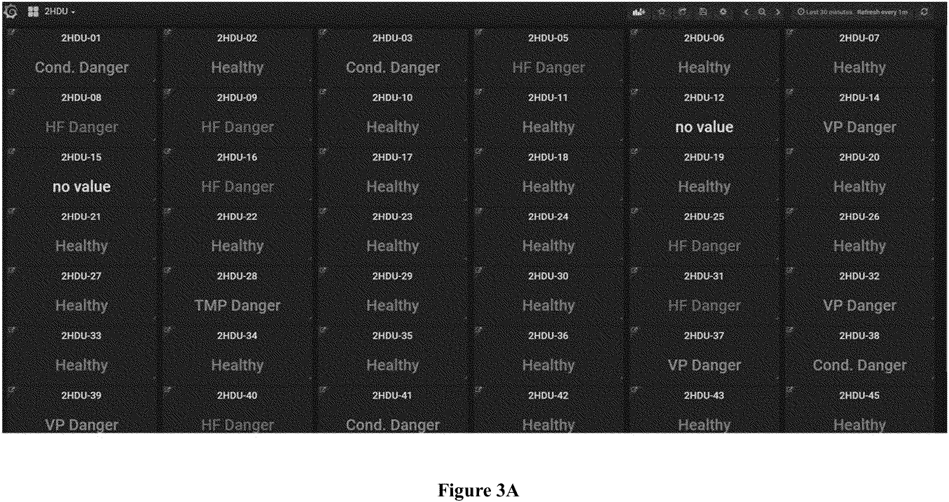

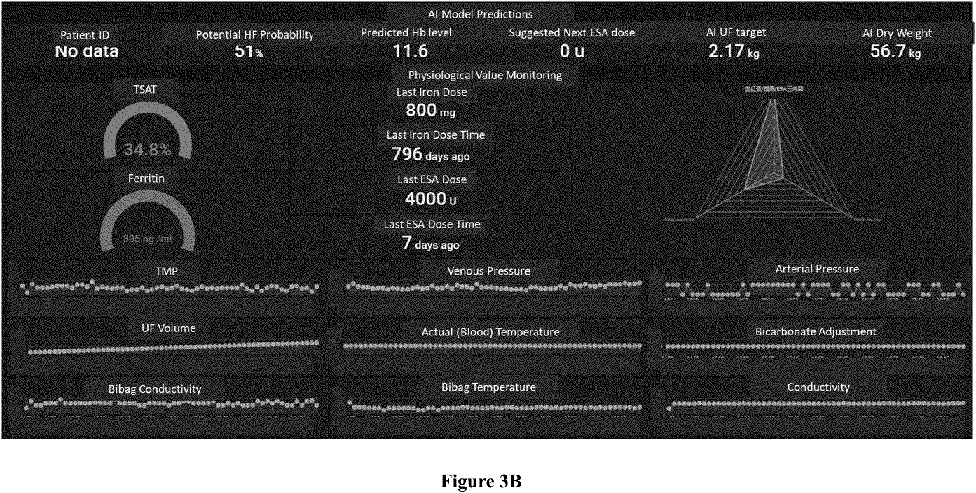

After predialytic and intradialytic data collection, the computation and construction of machine learning models were performed repeatedly during each hemodialysis session. The time-serial stream dialysis parameters of HD patients were collected, and the alarms were visualized on a Grafana dashboard when these parameters fluctuate outside of an established alarm limit on a main monitor screen (FIG. 3A). In addition, the personalized HF predictive values in the champion model and time-serial data were presented on another visualized Grafana dashboard (FIG. 3B).

### 2.4 Predictive Ability for HF

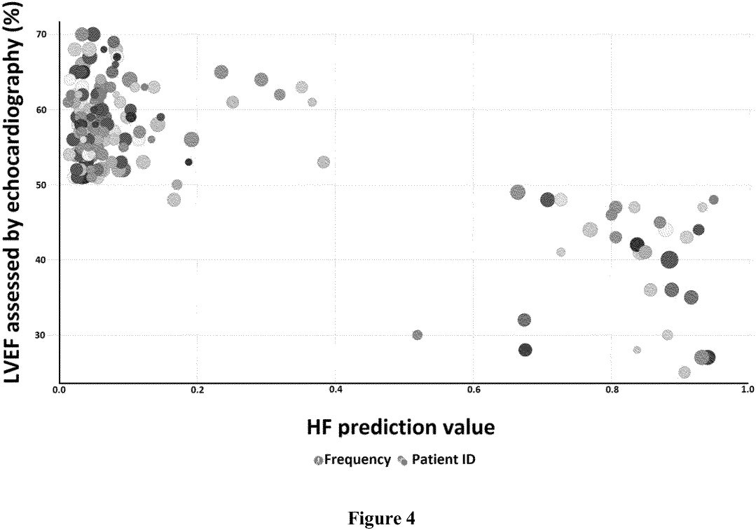

We presented a bubble plot to illustrate the correlation between LVEF as measured by echocardiography and the predictive ability for heart failure in our machine learning models. As shown in FIG. 4, the size of each bubble represents the number of hemodialysis sessions received by each patient. The position of the bubble vertically represents the predictive values for HF by machine learning models, and the position on the horizontal axis represents the corresponding LVEF, with good correlation between HF predictive values and LVEF values.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system, comprising:
a dialysis machine for performing a dialysis treatment in a patient; and
a computer system in communication with the dialysis machine, the computer system comprising:
one or more processors; and
a computer readable medium in communication with the one or more processors, the computer readable medium storing instructions that, when executed by the one or more processors, cause the computer system to perform:
inputting a plurality of first features and a plurality of second features of the patient to a first trained machine learning (ML) model,
wherein the plurality of first features is obtained during the dialysis treatment and the plurality of second features is obtained before the dialysis treatment,
wherein the plurality of first features includes a total ultrafiltration volume value and a total ultrafiltration time value received from the dialysis machine, and
wherein the plurality of second features includes a predictive value of pulmonary edema based on chest radiographic images, a Charlson comorbidity index value, a value of serum albumin level, a value of mean body surface area, a value of blood potassium level, and a value of predictive dry weight;
generating, using the first trained ML model, a result that indicates the risk of the patient for developing heart failure during the dialysis treatment; and
if the result indicates that the patient is at risk of heart failure, showing a warning sign on a display.

2. The system of claim 1, wherein the predictive value of pulmonary edema is generated using a second trained ML model with chest radiographic images of the patient as an input.

3. The system of claim 1, further comprising a time-series database which is connected to the computer system and stores real-time streaming intradialysis data received from the dialysis machine.

4. The system of claim 3, wherein at least part of the real-time streaming intradialysis data is shown on the display.

5. The system of claim 1, wherein the first trained ML model is trained using the following data:
intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and
predialysis data including demographic data, underlying comorbidities, containment medications and laboratory data.

6. The system of claim 1, wherein the value of predictive dry weight using a third trained ML model.

7. The system of claim 6, wherein the third trained ML model is trained using the following data:
intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and
predialysis data including demographic data, underlying comorbidities, containment medications and laboratory data.

8. One or more computer readable memories storing information to enable a computing device to perform a process comprising:
inputting a plurality of first features and a plurality of second features of the patient to a first trained machine learning (ML) model,
wherein the plurality of first features is obtained during the dialysis treatment and the plurality of second features is obtained before the dialysis treatment,
wherein the plurality of first features includes a total ultrafiltration volume value and a total ultrafiltration time value received from the dialysis machine, and
wherein the plurality of second features includes a predictive value of pulmonary edema based on chest radiographic images, a Charlson comorbidity index value, a value of serum albumin level, a value of mean body surface area, a value of blood potassium level, and a value of predictive dry weight;
generating, using the first trained ML model, a result that indicates the risk of the patient for developing heart failure during the dialysis treatment; and
if the result indicates that the patient is at risk of heart failure, showing a warning sign on a display.

9. The computer readable memories of claim 8, wherein the first trained ML model is trained using the following data:
intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and
predialysis data including demographic data, underlying comorbidities, containment medications and laboratory data.

10. The computer readable memories of claim 8, wherein the predictive value of pulmonary edema is generated using a second trained ML model with chest radiographic images of the patient as an input.

11. A system, comprising:
a dialysis machine for performing a dialysis treatment in a patient; and
a computer system in communication with the dialysis machine, the computer system comprising:
one or more processors; and
a computer readable medium in communication with the one or more processors, the computer readable medium storing instructions that, when executed by the one or more processors, cause the computer system to perform:
inputting a plurality of first features and a plurality of second features of the patient to a first trained machine learning (ML) model,
wherein the plurality of first features is obtained during the dialysis treatment and the plurality of second features is obtained before the dialysis treatment,
wherein the plurality of first features includes arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, transmembrane pressure, and
wherein the plurality of second features includes serum iron level, total iron binding capacity (TIBC), and transferrin saturation percentage (TSAT);
generating, using the first trained ML model, a predicted hemoglobin level of the patient during the dialysis treatment; and
showing the predicted hemoglobin level on a display.

12. The system of claim 11, further comprising a time-series database which is connected to the computer system and stores real-time streaming intradialysis data received from the dialysis machine.

13. The system of claim 12, wherein at least part of the real-time streaming intradialysis data is shown on the display.

14. The system of claim 11, wherein the first trained ML model is trained using the following data:
intradialysis data including arterial blood flow rate, effective blood flow rate, processed blood volume, dialysate flow rate, dialysate sodium level, dialysate sodium profile, dialysate temperature, dialysate bicarbonate level, dialysate conductivity, heparin volume, heparin bolus dose, heparin delivery rate, ultrafiltration rate, ultrafiltration volume, ultrafiltration time, arterial pressure, venous pressure, and transmembrane pressure, and
predialysis data including demographic data, underlying comorbidities, containment medications and laboratory data.
